(19) United States
(12) Patent Application Publication
(10) Pub. No.: US 2010/0204596 A1
(43) Pub. Date: Aug. 12, 2010

(54) METHOD AND SYSTEM FOR PROVIDING REMOTE HEALTHCARE

(76) Inventor: Per Knutsson, Vastra Frolunda (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 12/671,210

(22) PCT Filed: Sep. 17, 2008
(86) PCT No.: PCT/US08/76572

§ 371 Date: Jan. 29, 2010

Related U.S. Application Data
(60) Provisional application No. 60/973,427, filed on Sep. 18, 2007.

(51) Int. Cl. A61B 5/0402; G06Q 50/00; G06F 17/30

(52) U.S. Cl. 600/509; 705/2; 707/802; 707/769; 707/E17.044; 707/E17.014

(57) ABSTRACT

A method and system are adapted to provide healthcare to a patient. A portable registration device having a navigation function is provided. The registration device records health information of the patient. The health information is then automatically stored in a database that forms a digital medical journal. A medical professional activates the medical journal to retrieve the recorded health information. The medical professional prepares a diagnosis and treatment based on the health information stored in the on-line database.

### TECHNICAL FIELD

The present invention relates to a method and a system for providing remote healthcare.

### BACKGROUND AND SUMMARY OF THE INVENTION

A patient with a health problem normally must physically meet a healthcare provider such as a nurse or doctor. Very often the healthcare provider must ask routine questions and do a brief investigation of the patient's general health status before any diagnosis and treatment can be developed. The healthcare provider may also enter information into a medical/patient journal. All these steps are very cumbersome and take up unnecessary time both for the patient and for the healthcare provider. There is a need for a more efficient and secure method of obtaining healthcare.

The method of the present invention thus provides a solution to the above-outlined problems. More particularly, the present invention is a method and system for providing effective and efficient safe and convenient healthcare to a patient. The method provides thorough instructions to the patient or local medical professional about how to conduct the health care examination of the patient. The method provides a questionnaire with questions and instructions. A portable registration device may have a navigation function. The registration device records health information of the patient. In one embodiment, the navigation function locates an anatomical position of a virtual body of the patient corresponding to the health information recorded to store the health information at the anatomical position. The health information may be stored locally and/or be stored in on-line databases. The health information automatically forms the complete or partial digital medical journal of the patient. A medical professional may activate the medical journal in order to retrieve the recorded health information. The medical professional may then provide advice or prepare a diagnosis and treatment based on the health information stored in the on-line database of the digital medical journal or use the information as a complement to a physical examination/use as a follow up check of the evolution of the disease. Another important advantage is that the health information or medical journal may be accessed over the Internet so that it may be shared between the medical professionals and medical offices or hospitals.

One purpose of the present innovation is to gather and share digitalized medical information in an orderly and structured fashion and act as a virtual hospital. The system assists in the medical information exchange and to be an improvement and/or complement over conventional person-to-person meetings. The advantages of the system of the present invention is that the gathering period of the medical information may be extended in time and repeated several times. Additionally, the medical information may be stored and shared on a server in an orderly fashion. Also, the medical examination is, at least partly, executed by another person or machine than the medical expert, who in turn may deliver an opinion, advice, diagnose and or treatment.

The system and method of the present innovation is fed by multimedia information such as text, images, videos, sound or other information from different sources as long as these sources are digitalized. The sources that feed the system can be specially made for the system such as questionnaires to guide the examination. The system may also include specially designed medical instruments that communicates directly or indirectly to the system but also communicates with external sources such as examination instruments such as X-rays, laboratory tests and patient executed examinations that can be digitalized and recorded.

Another object of the system of the present invention is to gather information from different sources at different times to be examined at a later time. Another object is to deliver comparison means for a medical condition seen over time and possibly combined with one or several changes in treatments such as examining the blood sugar levels as the insulin level is changed over several weeks and to compare with values of healthy individuals.

### BRIEF DESCRIPTION OF DRAWINGS

FIG. 1 is a schematic view of the system of the present invention,

FIG. 2 is a schematic view of the information flow of the method of the present invention, and

FIG. 3 is a front view of a monitor showing a virtual body.

### DETAILED DESCRIPTION

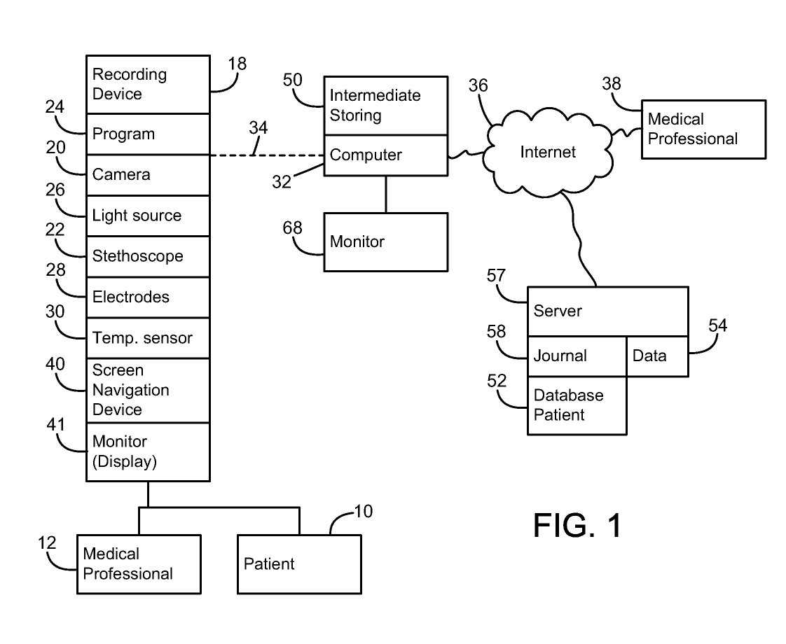

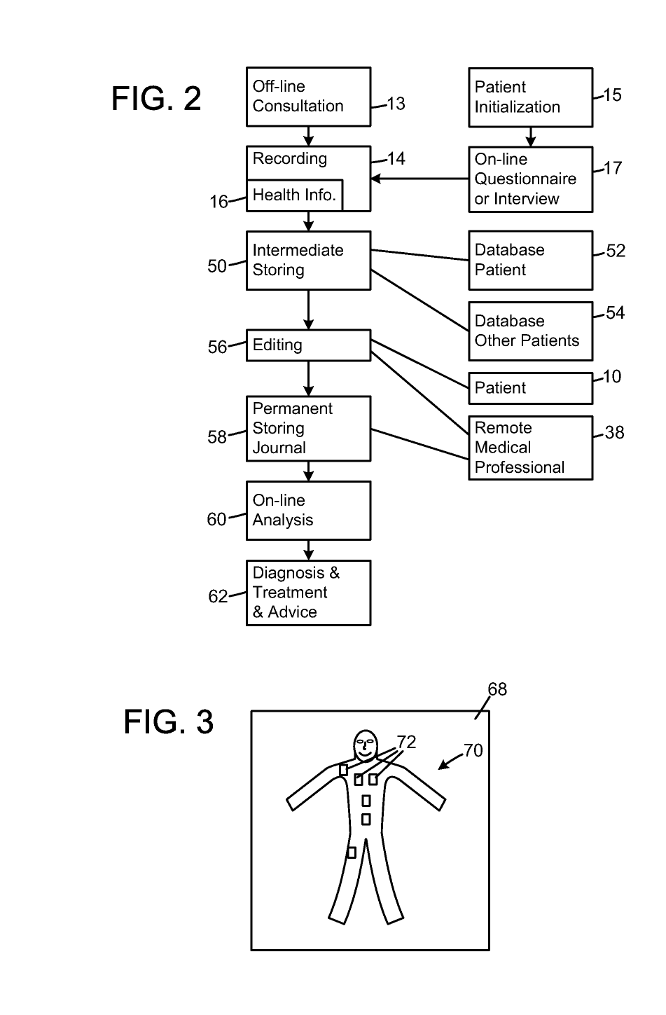

With reference to FIGS. 1-2, a patient 10 and a local medical professional 12 may first meet to conduct off-line consultation 13 by obtaining health information 16 of the patient 10. In the alternative, the patient or another person may conduct the examination. The health information 16 may be obtained with the assistance of a portable registration and recording device 18, and may be recorded in a recording step 14.

As explained below, this recorded health information 16 may be accessed on-line in real time by a health professional 38 or be viewed at a later time/date. The device 18 may, among other things, include a digital camera 20 and a digital stethoscope 22 that has a microphone. The camera may have an adjustable focus from less than 2 centimeters and up. If desired, the camera may also be used to take pictures closer than 2 centimeters. Preferably, the stethoscope may include a cavity that is covered by a membrane and from the cavity there is an air channel that leads to the built-in microphone. The device 18 may be a combination device that provides multi-functionality. However, it is also possible to use many separate and different registration devices. The system of the present invention may import data from external sources such as X-ray, laboratory results, ECG or any other source. It may also export data to other systems such as medical records databases.

It may also be possible for the patient 10 to initialize the medical consultation in an initialization step 15 by contacting a web page directly. The required health information may be provided to and used repeatedly by the patient. Automatic messages may also be sent to the medical professionals involved with the patient. It is also possible to provide structured instruction information to the patient. Additionally video conferences and telephone support may be used to provide instructions to the patient.

The patient will then be asked to answer an on-line computerized and interactive questionnaire 17 about the patient's standard personal data and questions related to the illness/problem of the patient. The interactive questionnaire asks intelligent questions to hone in and to be able to better focus on the patient's illness and to exclude unlikely causes. The questions asked to the patient depend on what answers the patient provides. The patient may also be instructed as to what and how the patient's health information 16 or physical data should be recorded by using the recording device 18 or by performing external investigations such as X-ray. The patient may then proceed with the recording step 14 of recording health-information according to the instructions. This recording may be done without the involvement of the medical professional. The transfer and recording of the health information may be automatic and be sent via the Internet, SMS, email or any other suitable communication channel. In this way, it may be possible to automatically analyze the recorded data by the recording device 18 and provide preliminary advice or diagnosis to the patient. The preliminary advice or diagnosis may either or both be based on answers provided by the patient to the questions of the questionnaire and/or automatic analysis of the physical recorded information of the patient's body obtained by the recording device 18. The information analyzed by the system may also be based on input provided by the patient such as the value of, for example, the blood pressure that has been recorded by the recording device. The system may also be directly linked to the recording device 18 so that the recorded data is automatically made available to the system for automatic analysis and diagnosis.

More particularly, the device 18 may also include a software program 24 that enables the taking of digital pictures and films with the camera 20 and the recording of sound from, for example, the heart and the lungs by using the stethoscope 22. The program 24 may also be used to store information about the historical and current physical health of family members and other users. The device 18 may include built-in light 26 so that the camera 20 can take clear pictures with correct colors and it is possible to take pictures of dark areas such as the mouth. The device 18 may also include a special adapter for the ear such as an otoscope. The device 18 may include electrodes 28, such as metal surfaces, that are connected to an amplifier to register ECG signals and other such information. The device 18 may also have a temperature sensor 30 for measuring a body temperature of the patient 10. The device 18 may also include a monitor 41 for depicting a virtual body of the patient that includes activatable and enlargeable anatomical positions, as explained below.

The device 18 may be connected to a computer 32 by wired or wireless communication 34 such as USB, Wifi or other communication technologies. The computer 32 may be connectable via the intranet or Internet 36 to a remote medical professional 38, such as a physician.

It is also possible for the patient or the local medical professional to use the device 18 to activate functions displayed on the screen of the computer 32. For example, the user of the device 18 may activate functions of the device by clicking on the functions displayed in the screen such as by clicking on a camera function on the computer screen to activate the camera function of the device 18. The user may thus use the device 18 to manipulate the monitor of the computer 32 to change the mode of the available functions of the device 18 such as changing from the stethoscope mode to the camera mode.

The program may include a code so that only authorized people may use the program 24 to gain access to the data on the device 18, the computer 32 or any other place such as a web server where the data may be stored.

The program may store information about the family structure of the patient and data related to the birth date, place, length, weight, social security number, name of parents, blood group etc. All input may automatically be time stamped.

Preferably, the device 18 has a plug and play program to activate the device 18 so that the user may view the information stored on the device 18 without having to install a program on the computer and gather new information on a standard personal computer.

As best shown in FIG. 2, once the off-line consultation 13 or the patient's own initialization 15 and response to the on-line questionnaire 17 are completed, the recording of physical health data or health information 16 of the patient, with the assistance of the recording device 18, may be initialized. It is also possible to import information from other sources such as X-ray, laboratories but also information from other medical professional such as therapeutic professionals so that different professionals may access information in the system and add new information. As indicated above, the questionnaire may ask routine medical questions to the patient that is outlined as a structured on-line interview such as questions related to medical history, allergies etc. and questions related to the medical problem at issue. The questionnaire step 17 may also provide instructions to the patient about how to use the device 18 and what digital physical data should be obtained. For example, the device 18 may be used to carry out measurements on the patients to gather physical information of the patient such as pulse, ECG, blood pressure, sound and temperature. The camera portion 20 may be used to take pictures of readings from other measurement instruments and pictures of body-portions the patient him/herself. For example, the camera 20 may be used to take a picture of the blood pressure gauge that shows the current blood pressure of the patient. It may be possible to use programs such as OCR programs that can read text and numbers from such instruments and interpret them. An OCR function may be included in the device 18. Screen shots, that have been generated by other external programs or examinations, that are displayed on the computer may be imported and edited.

All the recorded digital data and the data from the consultation 13 and/or the questionnaire 17 may then be stored in an intermediate storing step 50. The screen navigation function 40 of the device 18 may be used to control menus on the computer 32 in order to, for example, digitally store the recorded physical data or health information 16. The data in step 50 may be compared to information stored in at least two different databases. For example, a first database 52 may store historical data of the patient 10 and a second database 54 may store data of other patients with normal values or similar problems. An important feature is that the mouse 40 may be used to navigate on the computer 32 and on the monitor 68 thereof so that the recorded physical data and other information are stored in the correct anatomical position 72 on the patient's virtual body 70 depicted on the computer monitor 68, as shown in FIG. 3. Various gyro and camera devices may also be used for registering and recording the health information. For example, sound information from a left lung of the patient may be anatomically stored in a place that depicts the left lung. In this way, it is easy for a future reader to know which physical information is stored where on the computer. Similarly, sound signals from a right side of the heart are saved in a place that is associated with the right side of the patient's heart. This also reduces the risk of mixing up the recorded physical data or health information.

This may be done without contacting a medical professional or the service provider of the medical system of the present invention. This means the databases 52 may be temporarily stored on the device 18. This stored information may later be transferred by wireless, or wired communication, from the device 18 or memory card to the computer 32. This means the device 18 or its memory card could be used on any computer and it is not necessary that the computer has any special programs since all the plug and play programs may be included in the device 18.

In an editing step 56, the patient or the local medical professional may edit the information stored in the databases 52. It may also be possible to edit information stored in the device 18 prior to transferring the information to the databases 52, 54. Preferably, the stored information may then be permanently and chronologically stored in a permanent digital storage journal 58 on a server 57 by an approved medical professional such as a physician or another authorized medical professional. Preferably, the medical journal 58 stores the health information in a chronological order. The physician may then conduct an on-line analysis 60 of the stored information to develop a diagnosis 62 and plan for treatment. It may also be possible merely provide advice to the patient without developing a diagnosis. The medical profession may then retrieve information from the medical journal 58, as desired. The medical professional may move up, down and to the sides in the medical journal to find and retrieve information as desired. The medical professional may select different filters of information so that the medical professional only sees comments from a therapist, nurse or the comments from the nurse plus a physician or information provided during a certain time period for a certain illness. As an alternative to using the digital medical journal 58, the medical professional may, for example, listen to the sound of the right lung, by simply clicking on the right lung of the virtual body 70 of the patient that is shown and downloadable from the monitor 68 of the computer 32 or the server 57. The device 18 may also include a display or monitor 41 that shows the virtual body of the patient so that the patient can see that the data is saved in the correct anatomical position 72 of the patient's virtual body 70. If necessary, the anatomical position 72 may be enlarged for better clarity and details so that the medical professional to see the details better. The device 18 may include a gyro or any other similar positioning device so that the position of the device 18 may be monitored by the computer 32. The system may also include a video camera that records and shows where the device 18 is positioned on the body of the patient.

The information stored in the permanent storing journal 58 may also be accessed by the patient by using the Internet. It is also possible for the medical professional to maintain the medical journal 58 confidential by adding a password thereto. Different users may be given read and or write permissions.

An important aspect of the method of the present information is that all the necessary information for being able to diagnose and decide the correct treatment is already available to the medical professional in step 60 without requiring the medical professional to be in communication with the patient in real time. However, it may be possible for the medical professional to require the patient or local nurse to obtain additional information with the device 18, the questionnaire and any required additional tests such as X-ray or laboratory results.

It may also be possible to store information in a central database to, for example, identify epidemics and other socially or economically important information. This stored information may not necessarily identify the name of the patient but be more used for statistical purposes.

The health information stored in the on-line databases may serve as an information bank for health professionals. This means that when a patient adds information to the patient's own medical journal and the database is not shared by other patients, this database may be a source of information for everybody that works and assists the patient, such as a patient with multiple injuries from an traffic accident. Various medical professionals, specialists and others may read and add information to the medical journal by, for example, sending in videos, pictures and other ways of sending in health information about the patient to the online database. The medical professional may add arrows and mark texts to hone in on the correct advice and diagnosis to be delivered to the patient.

Of course, it is possible to add restrictions to the access to the sensitive information in the online medical journal in the database to prevent unauthorized use of the information. The online database at the website may also have a recording function so that each user must log in and register before any information is changed or added so that it can be determined who added or read what information and at what date and time. Also, different user groups, such as doctors, nurses and external consultants may have different colored text. The system may also be configured to send out messages to the various medical professional and others to schedule visits, testing, examination and other activities so that the system is similar to a company intranet for the parties involved with the particular patient concerned. The system may also send reminders to the patient to, for example, take the medicine at certain time intervals or visit the physician. It is only necessary to activate a reminder function of the system to trigger the reminders. It is also possible for the patient to monitor the patient's illness by answering simple questions asked by the system to better determine the status of the illness. A questionnaire about the patient's health status may periodically be sent to the patient. The patient may also be required to provide certain health information such as the blood pressure or blood sugar level and feed the system with this information into the medical journal so that the patient, doctor or nurse later can review the journal and obtain information of the status of the patient's illness.

The system of the present invention may be linked to an economical module that checks whether the patient 10 has paid for the services before permitting the patient to use the system.

While the present invention has been described in accordance with preferred compositions and embodiments, it is to be understood that certain substitutions and alterations may be made thereto without departing from the spirit and scope of the following claims.

1. A method for providing health consultation to a patient, comprising:
providing digitized instructions about how to conduct an examination of the patient (10),
providing a portable registration device (18),
providing a server (57) function for storing digitized health information in one or more of the following formats: picture, sound, video,
recording health information (16) of the patient (10) using the portable registration device (18) and/or a computer,
storing the health information (16) from the patient in a first database (52),
a medical professional (38) retrieving the health information (16) from the first database (52) and retrieving health information from a second database (54), the second database (54) containing stored information from other patients with normal values or from other patients having problems similar to problems of the patient,
the medical professional (38) conducting an on-line analysis (60) to establish a medical diagnosis based on the retrieved health information from the first database (52) and the second database (54) without being in communication with the patient in real time, and
the medical professional (38) permanently storing the health information (16) and the diagnosis (62) in a digital medical journal (58).

2. The method according to claim 1 wherein the method further comprises providing a patient initiating step (15) with an on-line questionnaire and interview (17).

3. The method according to claim 1 wherein the method further comprises transferring recorded health information (16) to databases (52, 54) on an Internet or intranet server (57).

4. The method according to claim 2 wherein the method further comprises the patient (10) or local caregiver editing the health information (16) stored in the preliminary database (50) before transferring to databases (52, 54).

5. The method according to claim 1 wherein the method further comprises a remote medical professional (38) permanently storing the health information (16) in the digital medical journal (58).

6. The method according to claim 1 wherein the method further comprises the remote medical professional (38) conducting an on-line analysis (60) of the health information (16) stored in the digital medical journal (58) without being in communication with the patient (10).

7. The method according to claim 1 wherein the method further comprises using a navigation function (40) to locate an anatomical position (72) of a virtual body (70) of the patient (10) corresponding to the health information (16) recorded, the patient storing the health information in the anatomical position (72) of the virtual body (70) of the patient.

8. A healthcare system for diagnosing a patient, comprising:
a portable recording device (18), the recording device (18) having a camera (20), a stethoscope (22) and a navigation function (40),
the recording device (18) being in communication with a computer (32),
means for recording health information (16) and storing the recorded health information in a correct anatomical position (72) of a patient's virtual body (70) displayed on a computer monitor (68) of the computer (32),
the computer (32) being in digital communication with a medical professional (38) via a network (36), and
means for retrieving the recorded health information by activating the anatomical position (72) displayed on the monitor (68).

9. The healthcare system according to claim 8 wherein the device (18) has a program (24) for recording the health information (16).

10. The healthcare system according to claim 8 wherein the device (18) has light source (26).

11. The healthcare system according to claim 8 wherein the device (18) has electrodes (28) for recording heart signals.

12. The healthcare system according to claim 8 wherein the device (18) has a temperature sensor (30) for measuring a body temperature.

13. The healthcare system according to claim 8 wherein the device (18) has a monitor (41).

14. The healthcare system according to claim 8 wherein the system has a server (57) in communication with the computer (32) and the medical professional (38).

15. The healthcare system according to claim 14 wherein the server has a permanently stored medical journal, the journal contains digitized health information in one or more of the following formats: text, data, picture, sound, video.

16. The healthcare system according to claim 8 wherein the computer has a monitor depicting a virtual body of a patient, the virtual body having activatable anatomical positions, and means for activating the anatomical positions on the monitor with the navigation function of the recording device to retrieve the recorded health information.
